(19) United States
(12) Patent Application Publication
(10) Pub. No.: US 2008/0300536 A1
(43) Pub. Date: Dec. 4, 2008

(54) Drug Mixing and Delivery Device

(76) Inventors: Xinming Wang, Guangdong Province (CN); Chunqing Jin, Guangdong Province (CN); Yan Liu, Guangdong Province (CN)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 11/721,932

(22) PCT Filed: Nov. 11, 2005
(86) PCT No.: PCT/CN05/01903

§ 371 Date: Jun. 15, 2007

(30) Foreign Application Priority Data
Dec. 16, 2004 (CN) 200420086385.X
Jan. 17, 2005 (CN) 200520078247.1
Apr. 27, 2005 (CN) 200520078680.5

(51) Int. Cl. A61M 5/31

(52) U.S. Cl. 604/89

(57) ABSTRACT

A medicine mixer for applying drug comprises a menstruum vial (12), an outer cannula (2) and a solute vial (11) (powdered drug ampoule) which are in one. Retaining ring (1), chuck ring (4) and convex ring (8) are disposed respectively at up portion, middle portion and nether portion of the inner wall of the outer cannula (2). An inner cannula (5) with a ducting needle (3) is disposed between the chuck ring (4) and the convex ring (8). In use the lower end of the outer cannula (2) is inserted to the opening (14) of the solute vial (11), and the opening (13) of the menstruum vial (12) is inserted into the retaining ring (1) of the outer cannula (2), so that rubber plugs (10,9) are pierced successively by the ducting needle (3) to connect two vials and thus mix drug. Then the outer cannula (2) is unfixed and the drug is applied to an infusion bottle (15). A medicine mixer for applying drug which can be repositioned automatically and a medicine mixer for applying drug which can delivery drug to many ampoules are also provided. The structure of the device is simple and cost is low. It is suitable to be combined with commercial ampoules. It is used conveniently and simply, and applied broadly.

### CROSS REFERENCES TO RELATED APPLICATIONS

This is a U.S. National Stage Application which claims the benefit of priority, under 35 U.S.C. §371, to International Application No. PCT/CN2005/001903, filed Nov. 11, 2005, which claims priority to Chinese Application No. 200420086385.X, filed on Dec. 16, 2004; Chinese Application No. 200520078247.1, filed on Jan. 17, 2005; and Chinese Application No. 200520078680.5, filed on Apr. 27, 2005.

### BACKGROUND OF THE INVENTION

1. Field of the Invention

The present invention relates to a drug mixing and delivery device, particularly to a drug mixing and delivery device having at least one pressurized vial. The drug mixing and delivery device can be automatically repositioned and can inject the contents of a solvent vial into a plurality of solute vials containing powdered drugs for reconstitution.

2. Description of the Related Art

Conventional methods for mixing at least two drugs or reconstituting a drug utilizing a syringe are cumbersome and inefficient. For example, when reconstituting a powdered drug, a nurse or medical personnel will first draw some water or solvent using an ordinary syringe to be injected into a vial containing a powdered drug. Once the drug is fully dissolved in solution, the mixture may then be withdrawn and injected into a separate vial for storage. This process is complicated, inefficient and risks contamination of the resulting drug mixture.

Conventional drug mixing and delivery devices that incorporate pre-sealed vials and/or cartridges have a complex structure that require the usage of customized vials and/or cartridges. These prior art devices are disadvantageous because they are not compatible with existing commercial vials commonly found in the market and are expensive to manufacture.

### SUMMARY OF THE INVENTION

Accordingly, it is an objection of the present invention to provide an efficient and effective drug mixing and delivery device that has a simplified structure is compatible with conventional commercial vials.

In order to achieve the above objectives and other objectives, a drug mixing and delivery device of the present invention includes an outer sleeve, an inner sleeve, a hollow needle and a pressurized solvent vial. The inner sleeve is inserted into the outer sleeve, wherein the inner sleeve and outer sleeve are movable relative to one another along the central axis of the sleeves. The needle extends through the center of the outer sleeve and inner sleeve along a center axis. A collar that engages with the mouth of the solvent vial is formed on a distal end of the inner wall of the outer sleeve. A flange is formed on a middle portion of the inner wall of the outer sleeve. A circular bulging portion is formed on a distal end of the inner wall of the outer sleeve. One end of the inner sleeve is sealed and a collar, having an inward bias, is formed on the inner wall of the inner sleeve near the open end. The inner sleeve is positioned between the flange and the circular bulging portion, with its open end pointing outward. The hollow needle extends through the inner sleeve along its center axis and is fixed to the inner sleeve in the center of the sealed end of the inner sleeve.

Preferably, the drug mixing and delivery device includes a collar having a cross-section shaped like a triangle and wherein an inner diameter of the flange is smaller than that of the collar and the circular bulging portion. Expansion joints are formed in the outer sleeve on the side engaging with the inner sleeve.

When the drug mixing or reconstitution is completed, the solute vial or the outer sleeve may be removed so that the needle withdraws from the rubber stopper of the solute vial.

The invention may further include an automatic repositioning means that would enable the needle to withdraw from the rubber stopper of a solute vial by itself. An automatic repositioning drug mixing and delivery device comprises an outer sleeve, an inner sleeve, a hollow needle, a elastic member and a pressurized solvent vial, in which the inner sleeve is inserted into the outer sleeve and is movable relative to the outer sleeve along a longitudinal central axis of the sleeves. The hollow needle pierces through the center portion of the outer sleeve and the inner sleeve along the central axis. A distance plate having a center hole is provided inside the outer sleeve and a distance piece is provided on the inner sleeve coupled to the solvent vial. One end of the hollow needle extends out of the distance piece of the inner sleeve and is fixed to the distance piece. An elastic member is provided between the distance plate of the outer sleeve and the distance piece of the inner sleeve; the outer sleeve and the inner sleeve are respectively provided with retaining members that engage with each other.

In a preferred embodiment, the end of the hollow, needle which extends out the distance piece of the inner sleeve, is provided with a protective sheath, while the other end of the hollow needle is positioned inside a thorough hole formed on the distance plate. Furthermore, the elastic member may be a spring or an elastic rubber sheath.

In a preferred embodiment, the distance piece is positioned inside the inner sleeve. A round bulge is formed on the inner wall of the inner sleeve at one end of the inner sleeve. The round bulge and the mouth of the solvent vial are tightly fitted or interference fitted with each other.

In a preferred embodiment, the distance piece is positioned at the top portion of the inner sleeve and the diameter of the distance plate is greater than that of the outer sleeve.

In a preferred embodiment, a round bulge is formed on the inner wall of the outer sleeve at one side. The round bulge and the mouth of the solute vial are tightly fitter or interference fitted with each other.

In a preferred embodiment, one side of the outer sleeve is provided with expansion joints along the axial direction, and a collar is formed on the inner wall of the outer sleeve. The distance between the outer sleeve and the distance plate is equals to or slightly greater than the thickness of the outer edges of the mouth of the solute vial.

In a preferred embodiment, the retaining members are sliding channels or open grooves having locking notches formed in opposite direction on the inner wall of the outer sleeve; clippers, formed on the outer wall of the inner sleeve, engage with the sliding channels or open grooves and the locking notches.

In clinical practice, to satisfy a standard dosage requirement, it is usually necessary to mix and transfer multiple vials worth of drugs, typically 3-5 vials, to a transfusion bottle. Because of this significant volume requirement, conventional methods require the usage of multiple drug mixing and delivery devices, which is wasteful and expensive.

Therefore, a drug mixing and delivery device for reconstituting powdered drugs contained in a plurality of solute vials is proposed. The device comprises an outer sleeve, a bush, an inner support, an inner sleeve, a hollow needle, elastic members and a pressurized solvent vial. The inner sleeve is inserted in the outer sleeve and movable relative to the outer sleeve along a longitudinal central axis of the sleeves. The hollow needle pierces through the central portion of the outer sleeve and the inner sleeve along the central axis. The outer sleeve is connected to the bush provided with a movable plate. The elastic members are provided above and below the movable plate respectively. The movable plate is confined within the bush by a collar. The inner support is positioned within the outer sleeve. The hollow needle is fixed to the movable plate and is positioned inside a thorough hole formed in the inner support and a thorough hole formed in the bush. An end cap is connected to the outer sleeve via a ripping ring. The inner sleeve is inserted into the end cap.

Preferably, in the drug mixing and delivery device for reconstituting powdered drugs contained in a plurality of solute vials of the invention, the upper portion of the inner sleeve engages with the mouth of the solvent vial and the lower portion of the bush engages with the mouth of the solute vial. An annular step or a bulge is formed on the upper portion of the inner sleeve. The maximum traveling distance of the inner support is defined by an annular step formed inside the outer sleeve. The elastic member can be a a spring or an elastic rubber sheath.

The drug mixing and delivery device of any embodiment of the present invention for reconstituting powdered drugs contained in a plurality of solute vials can distribute the contents of a pressurized large volume solvent vial to a plurality of solute vials containing powdered drugs. Then the pressurized drug mixture contained in these vials can be delivered to a transfusion bottle one by one utilizing the same drug mixing and delivery device. Therefore, due to the simple operation of this inexpensive device, the drug mixing and delivery device of the present invention is suitable for clinical use.

### BRIEF DESCRIPTION OF THE DRAWINGS

FIG. 1 is a vertical cross-section view of the drug mixing and delivery device of the present invention;

FIG. 2 is a planar view of FIG. 1;

FIG. 3 is a vertical cross-section view of the drug mixing and delivery device of the present invention in which the contents of a solvent vial and a solute vial are being mixed.

FIG. 4 is a vertical cross-section view of a solvent vial and a solute vial mounted on the drug mixing and delivery device of the present invention prior to initiating mixing.

FIG. 5 is a vertical cross-section view of the drug mixing and delivery device of the present invention showing the solute vial and the inner sleeve without the outer sleeve.

FIG. 6 is a vertical cross-section view showing the drug mixing and delivery device of the present invention delivering a mixed drug solution to a bottle.

FIG. 7 is a vertical cross-section view of a solvent vial and a solute vial mounted on the automatic repositioning drug mixing and delivery device of the present invention prior to initiating mixing.

FIG. 8 is a vertical cross-section view showing the automatic repositioning drug mixing and delivery device of the present invention in which the contents of a solvent vial and a solute vial are being mixed.

FIG. 9 is a vertical cross-section view showing the automatic repositioning drug mixing and delivery device of the present invention without a solvent vial.

FIG. 10 is a partial perspective view showing a sleeve of the automatic repositioning drug mixing and delivery device of the present invention.

FIG. 11 is a planar view showing the inner sleeve of the automatic repositioning drug mixing and delivery device of the present invention.

FIG. 12 is a vertical cross-section view showing a variation of the inner sleeve of the automatic repositioning drug mixing and delivery device of the present invention.

FIG. 13 is a is a vertical cross-section view showing a variation of the inner sleeve of the automatic repositioning drug mixing and delivery device of the present invention.

FIG. 14 is a is a vertical cross-section view showing a variation of the inner sleeve of the automatic repositioning drug mixing and delivery device of the present invention.

FIG. 15 is a is a vertical cross-section view showing a variation of the inner sleeve of the automatic repositioning drug mixing and delivery device of the present invention.

FIG. 16 is a vertical cross-section view showing a variations of the inner sleeve of the automatic repositioning drug mixing and delivery device of the present invention.

FIG. 17 is a vertical cross-section view showing the automatic repositioning drug mixing and delivery device of the present invention with a solvent and solute vial.

FIG. 18 is a vertical cross-section view showing the automatic repositioning drug mixing and delivery device of the present invention without the solute vial.

FIG. 19 is a vertical cross-section view of one of many solute vials and a solvent vial that can be mounted on the drug mixing and delivery device for reconstituting powdered drugs prior to initiating mixing;

FIG. 20 is a vertical cross-section view of the drug mixing and delivery device for reconstituting powdered drugs in which the contents of one of may solute vials is being mixed with the contents of a solvent vial.

FIG. 21 is a vertical cross-section view of the drug mixing and delivery device for reconstituting powdered drugs in which a mixed drug solution is being transferred to a transfusion bottle.

### DESCRIPTION OF THE PREFERRED EMBODIMENT

The present invention is directed to a drug mixing and delivery device and a method for using said device that enables simplified and efficient mixing and delivery of a drug composition. The drug mixing and delivery device utilizes the internal pressure differential between two vials to transfer the contents of one vial into a second vial, wherein the vials may be any standard commercial drug vials or drug ampoules.

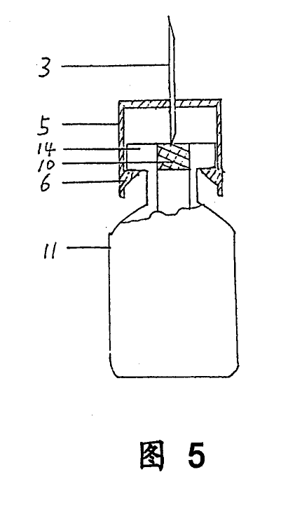

As shown in FIG. 3 and FIG. 4, the drug mixing and delivery device of the present invention generally comprises a solvent vial 12 and a solute vial 11 connected by a sleeve portion. Solvent vial 12 is inverted such that its mouth 13 is inserted into an upper distal end of an outer sleeve, 2 and a mouth 14 of solute vial 11 is inserted into a lower distal end of outer sleeve 2.

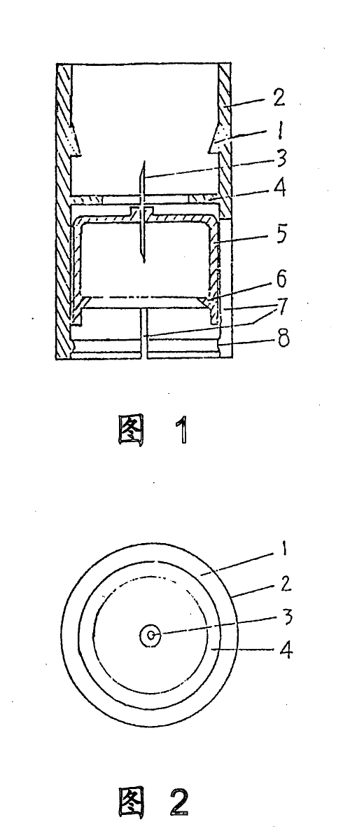

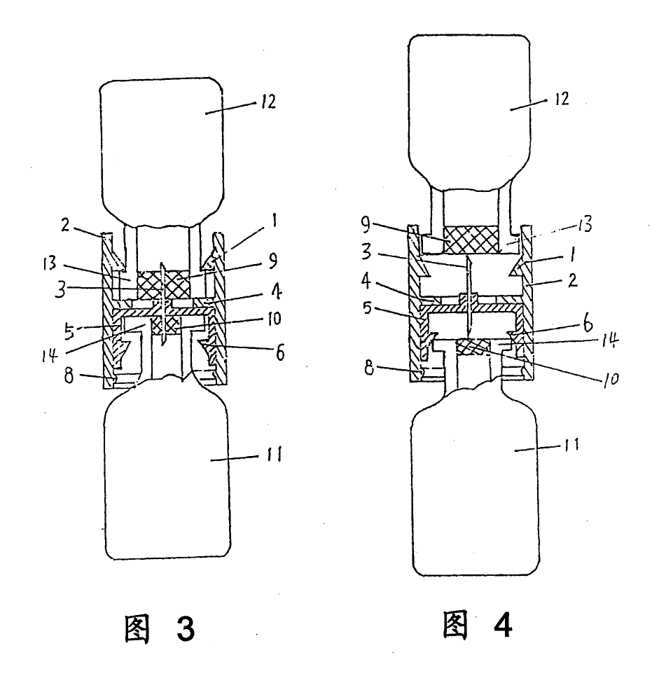

As shown in FIG. 1 and FIG. 2, a collar 1, which has a triangle-shaped cross-section, flange 4, which has a rectangle-shaped cross-section, and a circular bulging portion 8 are respectively formed on an upper, middle and lower portion of the inner wall of outer sleeve 2 of the drug mixing and delivery device of the present invention. A plurality of expansion joints 7 are formed longitudinally between flange 4 and the bottom edge of outer sleeve 2. Inner sleeve 5, which is positioned in the lower portion of an inner wall of outer sleeve 2, is similar to a bottle cap in its structure. A rounded collar 6, which is inwardly biased so as to securely engage a vial is formed along a bottom edge of inner sleeve 5 to engage with the bottom edge of mouth 14 of solute vial 11. A hollow needle 3 is fixed at the center of the inner sleeve along the central axis.

When using the drug mixing and delivery device of the present invention, first mouth 14 of solute vial 11 is inserted into collar 6, which is formed on inner sleeve 5, so that the hollow needle 3 pierces through a rubber stopper 10 of solute vial. Mouth 13 of solvent vial 12 may then be inserted into collar 1, which is formed on an inner wall of outer sleeve 2, so that the needle 3 pierces through rubber stopper 9 of solvent vial 12, thus initiating the drug mixing operation, as shown in FIG. 3.

Alternatively, solvent vial 12 may be previously assembled with the sleeve portion as part of a whole assembly. Similar to the previously disclosed method, needle 3 may either consecutively or simultaneously pierce stoppers 9 and 10 of solute vial 11 and solvent vial 12. During the manufacturing process, mouth 13 of solvent vial 12 may engage outer sleeve 2 until mouth 13 is wedged between the protrusion of collar 1, as shown in FIG. 4. Collar 1 functions as a positioning point that prevents needle 3 from penetrating stopper 9. Solvent vial 12 and outer sleeve 2 may be fixedly positioned relative to one another such that mouth 13 is wedged between the protrusion of collar 1, as shown in FIG. 4, and can be packed as an assembly for clinical applications. When in use, a nurse or medical personnel need only apply a slight force to insert solute vial 11 into the other side of outer sleeve 2 so that mouth 14 of solute vial 11 is properly engaged with inner sleeve 5 so as to be positioned to initiate drug mixing, as shown in FIG. 4.

Mixing may begin by applying force to inverted solvent vial 12, such as by pushing down in solvent vial 12. It will be appreciated that the force applied to solvent vial 12 will also be transferred to collar 1. Because flange 4 and outer sleeve 2 are formed as an integral structure and because flange 4 also engages with an upper portion of inner sleeve 5, the force applied to solvent vial 12 will also force outer sleeve 2 and inner sleeve 5 to move downward, and thereby force mouth 14 of solute vial 11 to be fully inserted within collar 6 of inner sleeve 5.

When the mouth 14 of solute vial 11 contacts the portion of inner sleeve 5 adjacent to flange 4, a lower end of needle 3 will pierce through rubber stopper 10, such that solute vial 11 becomes directly connected to needle 3, and such that collar 6 of inner sleeve 5 is positioned beneath mouth 14 of solute vial 11. A downward force may then be applied to solvent vial 12 until an upper end of needle 3 pierces through rubber stopper 9 of solvent vial 12, such that solvent vial 12 also directly connects to needle 3. Then the content of solvent vial 12 may be immediately injected into solute vial 11 through needle 3 since the internal pressure of solvent vial 12 is greater than that of solute vial 11. Therefore, the powdered drugs inside solute vial 11 may be dissolved in or fully mixed with the contents of solvent vial 12. Solvent vial 12 has already been pre-pressurized during the manufacturing process, so when it is connected to solute vial 11 through needle 3, the pressure differential between the vials will force the contents of solvent 12 into solute vial 11 until the pressure within the two vials reaches equilibrium, as shown in FIG. 3.

Because the distance between collar 1 and flange 4 is approximately equal to or slightly greater than the thickness of mouth 13 of solvent vial 12, mouth 13 may be tightly snapped into and secured between collar 1 and flange 4. After the contents of vials 11 and 12 are properly mixed, the outer sleeve of solvent vial 12 may be removed. As the solvent vial 12 is pulled upward from the sleeve, outer sleeve 2 will also move upward because mouth 13 of solvent vial 12 remains engaged with collar 1. At the same time, inner sleeve 5 will also move upward because circular bulging portion 8, formed on outer sleeve 2, engages with the collar 6, formed on a bottom portion of inner sleeve 5, until collar 6 of inner sleeve 5 engages with the bottom face of mouth 14 of solute vial 11. At this point inner sleeve 5 is in its highest position but does not disengages with solute vial 11, and a lower end of needle 3 is pulled out of rubber stopper 10. by continuously pulling solvent vial 12 upward, circular bulging portion 8 will move upward along the outer wall of inner sleeve 5 because of expansion joint 7 until outer sleeve 2 disengages with inner sleeve 5 completely. At this point needle 3 is positioned above mouth 14 of solute vial 11 because collar 6 formed on a lower portion of inner sleeve 5 tightly retains the lower edges of mouth 14 of solute vial 11, forming a pressurized automatic syringe, as shown in FIG. 5.

Alternatively, the above procedure can be performed by holding and pulling outer sleeve 2 in an upward direction to achieve the same effect and result.

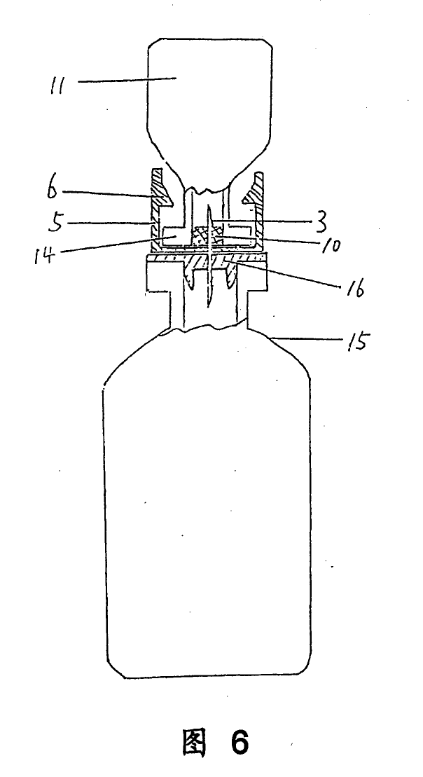

Solute vial 11 may then be turned upside down to transfer the mixed drug of solute vial 11 to transfusion bottle 15. As shown in FIG. 6, when one end of needle 3 pierces stopper 16 of transfusion bottle 15, a counteractive force will push needle 3 through rubber stopper 10 of solute vial 11. Because the pressure within solute vial 11 is greater than that of transfusion bottle 15, the contents of solute vial 11 will be injected into transfusion bottle 15 to complete a one-time drug delivery operation.

In addition, in order to better engage the mouths of solvent vial 12 and solute vial 11, outer sleeve 2 can have different customized inner diameters by forming a step in the middle of outer sleeve 2.

The drug mixing and delivery device of the present invention has many advantages. Not only can the drug mixing and delivery procedure be quickly executed, it also avoids possible contamination of the mixture by eliminating the need for multiple transfers of the drug solution and by eliminating usage of a conventional syringe.

Additionally, a portion of needle 3 located within the inner sleeve can be provided with an elastic rubber sheath to protect needle 3 from being contaminated. The rubber sheath will extend automatically to cover the needle end after the drug is delivered to protect the operator from accidental injury.

Additionally, because a special manufacturing means are necessary to pressurize solvent vial 12, the drug mixing and delivery device of the present invention is desirably constructed to be an environment friendly and disposable one time use appliance.

Another feature of the drug mixing and delivery device of the present invention is that the three sections, solute vial 11, solvent vial 12 and the connecting sleeve, can either be individually packaged in aseptic packages, or solvent vial 12 and the sleeve portion can be assembled and packed together. Alternatively, all three sections can be assembled together in the factory and packed in one aseptic package to facilitate the operation of the device and eliminate the possibility of mixing the wrong drugs.

As shown in FIG. 7, the drug mixing and delivery device of the present invention may further include a means for automatically returning or automatically repositioning the device to an original state. An automatic repositioning drug mixing and delivery device of the present invention generally comprises an outer sleeve 22, an inner sleeve 25, a needle 23, a elastic member 210, a solvent vial 12 and a solute vial 11. A distance piece 29 is formed inside inner sleeve 25 and is transversely oriented with respect to inner sleeve 25. A plurality of spaced round bulges 215 are formed on an inner wall of inner sleeve 25 above distance piece 29. Two clippers 21, formed at a lower end portion of an outer wall of inner sleeve 25, are symmetrically situated and protrude outward from inner sleeve 25. A distance plate 20 is formed inside outer sleeve 22 and is transversely oriented with respect to outer sleeve 22. A plurality of spaced round bulges 28 are formed on an inner wall of the outer sleeve 22 below the distance plate 20. As shown in FIG. 10, a pair of channels 24 is symmetrically formed in an inner wall of outer sleeve 22. Two locking notches 26 are formed at the distal ends of the pair of channels 24, facing in opposite directions with respect to each other, as shown in FIG. 10. The two clippers 21, formed at a lower end portion on the outer wall of inner sleeve 25, can be inserted into and moved along channels 24 and engaged with locking notches 26. Needle 23 having two piercing ends, is fixed to distance piece 29 of inner sleeve 25. One end of needle 23 extends out of the distance piece 29, and the other end of the needle 23 is positioned inside a through hole 212, formed in the center of distance plate 20 of outer sleeve 22. In a preferred embodiment, elastic member 210 is a spring which is positioned around needle 23 and extends between distance piece 29 and distance plate 20. The outer edge of mouth 13 of pressurized solvent vial 12 is tightly fitted and secured between round bulges 215, formed on the inner wall of inner sleeve 25, and the outer edge of mouth 14 of solute vial 11 is tightly fitted and secured between round bulges 28 formed on an inner wall of outer sleeve 22. FIG. 7 shows the placement of solvent vial 12 and solute vial 11 prior to mixing.

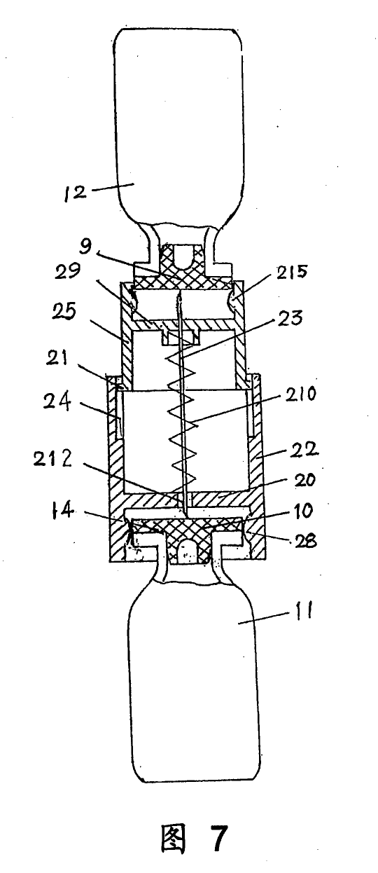

To initiate mixing the contents of the two vials, solvent vial 12 is pressed downward; inner sleeve 25 will then correspondingly move downward against spring 210. Clippers 21 will slide downward along channel 24 until they reach locking notches 26, while an upper end of needle 23 will pierce through rubber stopper 9 of solvent vial 12. When clippers 21 reach locking notches 26, the lower end of needle 23 will pierce through rubber stopper 10 of solute vial 11 so that needle 3 operatively connects the two vials and initiates mixing. The pressurized content of solvent vial 12 will then flow into the solute vial 11 through needle 23, as shown in FIG. 8. Solute vial 11 will then contain the pressurized mixed drug solution.

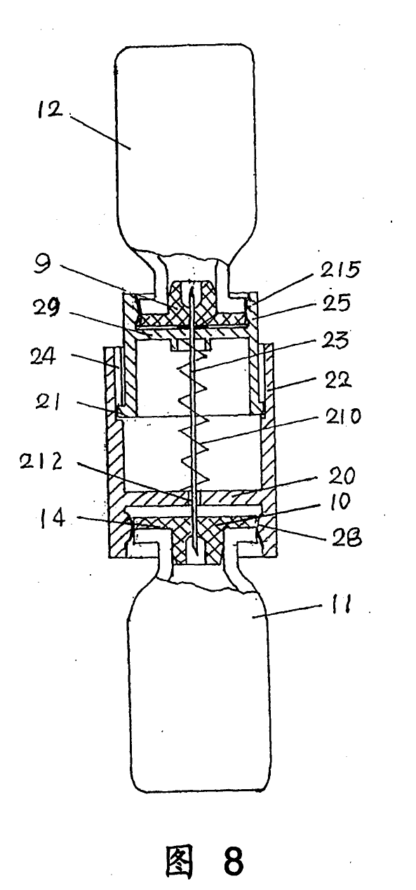

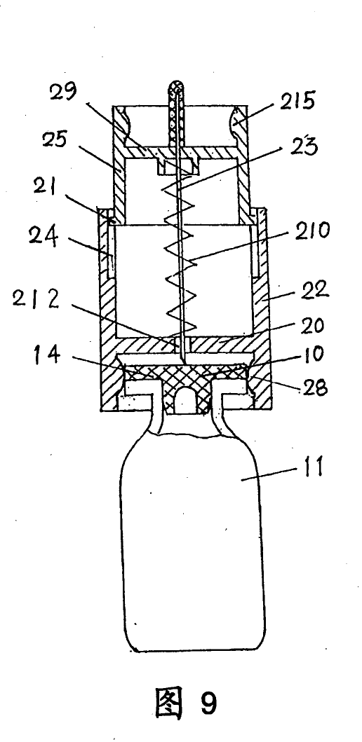

When mixing is complete, inner sleeve 25 may be loosened and returned to its original position; the elastic member 210, which is preferably a spring, will push against inner sleeve 25 when an initial external downward force to solvent vial 12 has been removed, as shown in FIG. 9. At the same time, the lower end of needle 23 will retract from rubber stopper 10 of solute vial 11. Solvent vial 12 may then be removed, and solute vial 11 may be turned upside down. Then an end of needle 23 that extends through distance piece 29 may be used in turn to pierce a rubber stopper of a transfusion bottle. Applying an external force against solute vial 11, clippers 21 of inner sleeve 25 will again slide along working channels 24 to locking notches 26. Due to a resultant counterforce, needle 23 will again pierce through rubber stopper 10 of solute vial 11. When needle 23 has pierced both stoppers, the mixed drug solution will be injected into the transfusion bottle due to the higher internal pressure within solute bottle 11, thereby completing a one-time delivery to the transfusion bottle, wherein the drug mixing and delivery operation are performed under aseptic condition.

Locking notches 26 retain the connection between solvent vial 12 and solute vial 11 by slightly rotating inner sleeve 2 clockwise so that clippers 21 latch with locking notches 26. Of course, locking notches 26 are an optional feature of the invention since the connection between solvent vial 12 and solute vial 11 can be retained simply by applying and maintaining pressure to solvent vial 12 or inner sleeve 25, i.e. by applying pressure with one's hand. Needle 23 will disengage with rubber stopper 10 once the pressure is released. Of course, other means can be used for retaining the connection between the two vials, such as a retaining ring, a protruding ring or a positioning step, etc. Elastic member 210 can also be a sleeve made of elastic rubber instead of a spring.

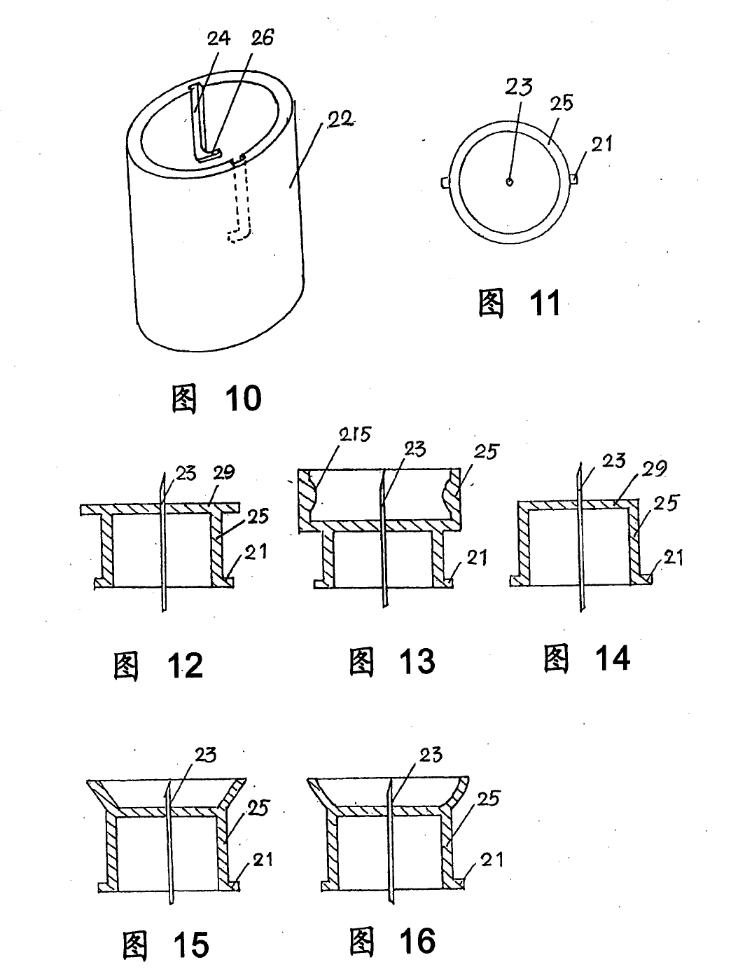

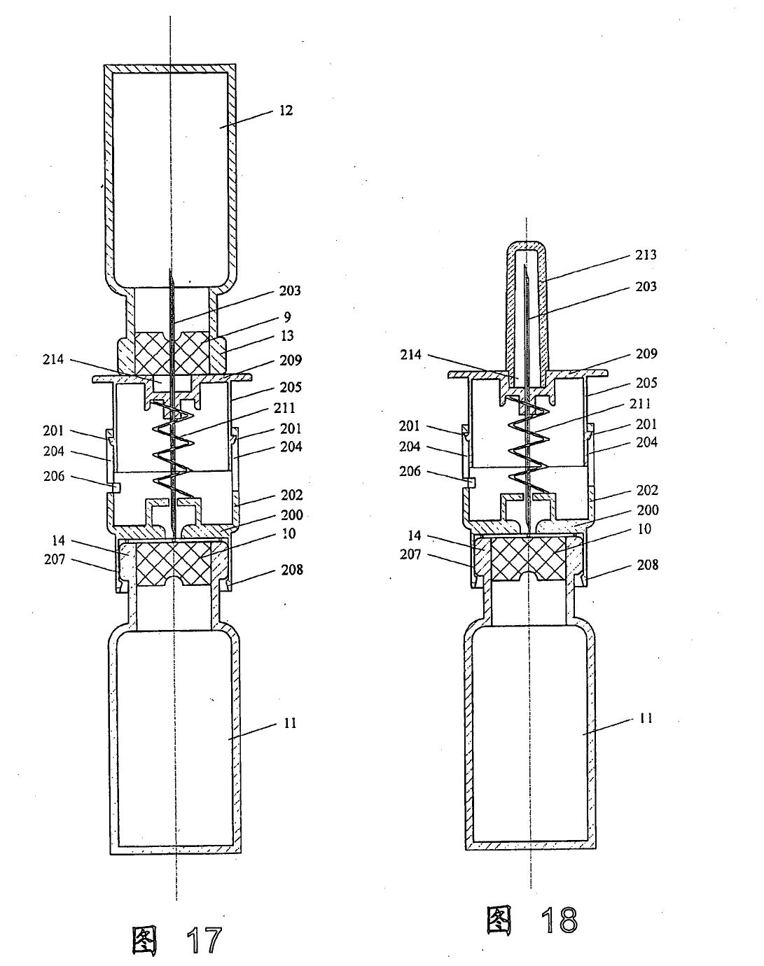

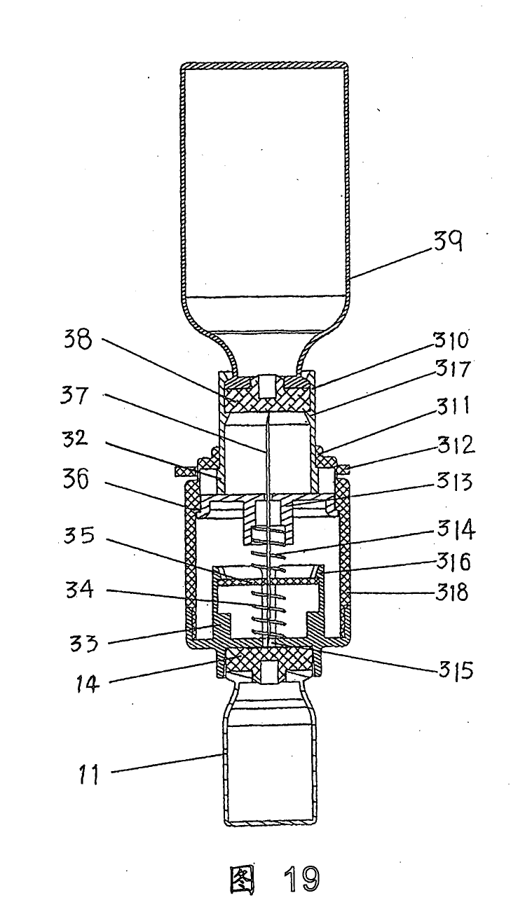

FIG. 12-FIG. 16 show various possible structural configurations of a distal end of inner sleeve 25 for mating with a solvent vial.

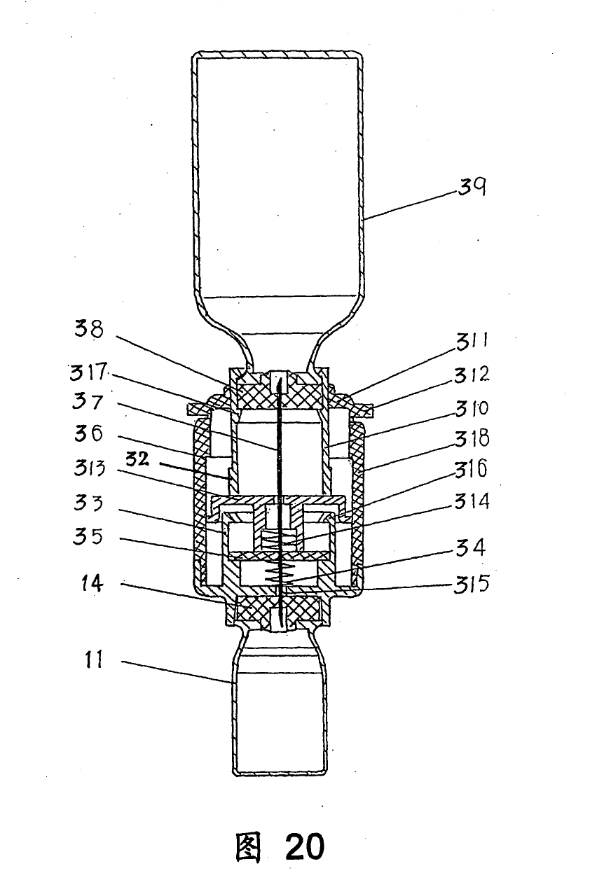

FIG. 17 and FIG. 18 show another embodiment of the automatic repositioning drug mixing and delivery device of the present invention. The difference between the embodiments of FIGS. 17-18 and FIGS. 7-8 is that the structural element above distance piece 29 of FIGS. 7-8 has been removed so that the mating ends of an inner sleeve 205 for receiving a solvent vial 12 is shaped like a flat plane, similar to the inner sleeve of FIG. 12. Two open grooves 204 and two open locking holes 206 engage with the two clippers 201, which are formed on inner sleeve 205. The mating ends of the outer sleeve 202 for receiving mouth 14 of solute vial 11 is shaped like a socket. The round bulge formed on the outer sleeve 202 is located at an edge of the socket, forming collar 208. The side wall of the socket is formed with a plurality of vertical, symmetrically spaced expansion joints 207. When solvent vial 12 is disengaged with inner sleeve 205, the protruding end of the needle 203 can be covered with a protective sheath 213 made of a hard material. A distal end of sheath 213, having an opening, is inserted into a recess portion 214, which is formed in the center of distance piece 209, to protect needle 203 from contamination or damage and to prevent accidental needle related injuries.

As shown in FIG. 19, a drug mixing and delivery device for a plurality of powdered drug vials of the present invention generally comprises an outer sleeve 318, a bush 33, an inner support 313, an inner sleeve 310, a hollow needle 37, a spring 314, a spring 34 and a large solvent vial 39. Bush 33 is fixed to a distal end of the outer sleeve 318, and an end of bush 33 includes a movable plate 35 through which extends needle 37. Spring 34 is set between movable plate 35 and a bottom portion of bush 33. The maximum travel distance of movable plate 35 within bush 33 is defined by collar 316, which is formed on an inside wall of an upper portion of bush 33. The inside of outer sleeve 318 is also provided with an inner support 313 having a needle hole. Spring 314 is set between an inner support 313 and a movable plate 35. The maximum travel distance of inner support 313, which is located inside outer sleeve 318, is defined by an annular step 36. An upper distal end of outer sleeve 318 is provided with an end cap 311 coupled to outer sleeve 318 through ripping ring 312. An annular step 317 formed on the inside of inner sleeve 310 forms the receiving socket for solvent vial 39. The end cap 311 engages with a step 32 formed on a lower portion of inner sleeve 310 so that inner sleeve 310 cannot be separated from outer sleeve 318 by pulling inner sleeve 310 in an upward direction.

When the two vials are not directly connected by needle 37, spring 34 pushes movable plate 35 upward until it reaches collar 316, and spring 314 pushes inner support 313 upward until it reaches annular step 36, which is located inside outer sleeve 318. This configuration represents an initial position of the device prior to an application of external force on the device or after releasing an external force from the device.

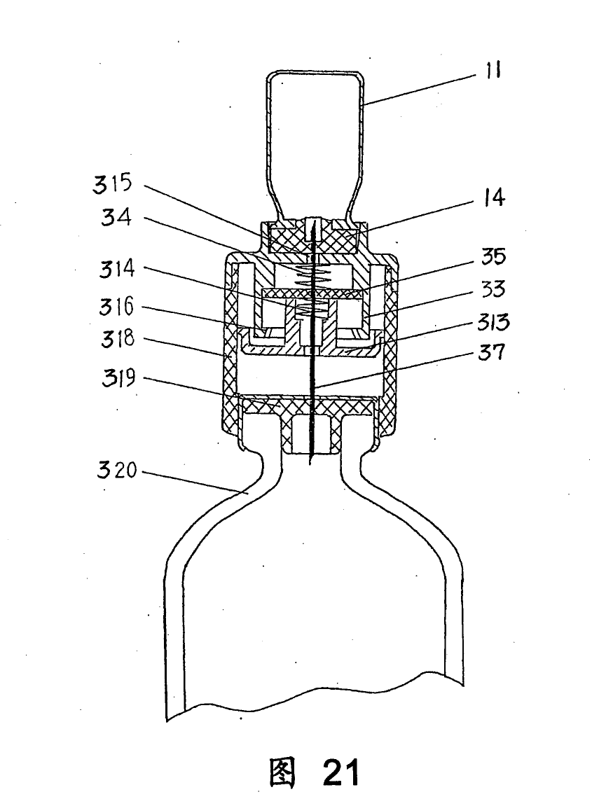

During drug mixing, solvent vial 39 may be pushed downward with a little force. Stopper 38 of solvent vial 39 will press against annular step 317 so that inner sleeve 310 presses against inner support 313 accordingly. Movable plate 35 will then be pressed by compressed spring 314, which in turn is pressed by inner support 313. Meanwhile, needle 37 will pierce through stopper 14 of solute vial 11 through a thorough hole 315, and at the same time, needle 37 will pierce stopper 38 of solvent vial 39, so that the two vials are directly connected via needle 37, as shown in FIG. 20. Because graduated solvent vial 39, which may have a plurality of marks indicating volume, is pressurized, the contents of solvent vial 39 will flow into solute vial 11 via needle 37. Solvent vial 39 may be released when a predetermined amount of the content in solvent vial 39 is delivered to a solute vial 11.

The above operation can be repeated so that the content of solvent vial 39 can be introduced to several solute vials 11 that may contain the same or different drugs and thereby pressurize multiple solute vials 11. Only one drug mixing and deliver device is necessary to deliver the drug solution from solute vials 11 to a transfusion bottle.

During the drug delivery operation for delivering the mixed drug solution in solute vials 11 to transfusion bottles, ripping ring 312 may be removed by hand so that end cap 311, inner sleeve 310, solvent vial 39 and outer sleeve 318 are separated. Then, as shown in FIG. 21, outer sleeve 318 and solute vial 11 are inverted so that outer sleeve 318 covers a mouth of transfusion bottle 320. Pressure is then applied to solute vial 11 so that the parts comprising outer sleeve 318 assume the positions depicted in FIG. 20. At this time, needle 37 pierces through stopper 319 of transfusion bottle 320 and the contents of solute vial 11 is injected into transfusion bottle 320. Solute vial 11 may be replaced with another solute vial 11 to repeat the above operation so that the contents of several solute vials are delivered to one transfusion bottle 320.

Only one drug mixing and delivery device is necessary to distribute the contents of a solvent vial into several solute vials, which contain powdered drugs, thereby pressurizing said solute vials so that it is possible to deliver the mixed drug solutions from multiple solute vials to a transfusion bottle. This will reduce the number of the drug mixing and delivery device required in a large scale operation and facilitate operation as well as reduce cost.

### INDUSTRIAL APPLICABILITY

To operate the drug mixing and delivery device of the present invention, one need only insert the mouth of a solvent vial into a corresponding mating portion of the device and insert a solute vial into a corresponding opening in an inner sleeve of the device. By applying an external pressure to the solvent vial, the ends of a hollow needle pierce through the rubber stoppers of said vials. Because the solvent vial is pressurized, its contents will flow into the solute vial to mix with the contents of the solute vial. After mixing, the solvent vial may be separated from the outer sleeve of the device by pulling on the solvent vial. The remaining solute vial, engaged with the inner sleeve of the device, essentially functions as a pressurized syringe. The mixed drug solution may then be transferred from the solute vial to a transfusion bottle by allowing the hollow needle to pierce through the rubber stopper of the transfusion bottle and solute vial. The simple drug mixing and delivery device of the present invention significantly reduces the possibility of contamination and improves work efficiency.

In addition to the features mentioned above, the drug mixing and delivery device can be automatically repositioned. After the drug mixing operation is completed, the hollow needle will move out from the rubber stopper of the solute vial and the solute vial will be automatically reverted to its original sealed condition upon releasing the force applied by an elastic member. When the volume of the solvent vial and the solute vial is large, requiring a long period of time for drug mixing, it is possible to maintain the connection between the two vials by engaging a pair of clippers, located on the outer wall of the inner sleeve, with the locking notches of the device by applying pressure to the solvent vial until the needle pierces the stopper of the solvent vial and then rotating the inner sleeve so that the clippers lock with the locking notches. When the drug mixing operation is completed, the clippers may be disengaged from the locking notches by rotating the inner sleeve in a reverse direction; the hollow needle will then retract from the rubber stopper of the solute vial.

The drug mixing and delivery device may also be used to reconstituting drugs contained in several solute vials drugs and can be used to distribute the pressurized contents of a large solvent vial to several solute vials. The contents of the multiple solute vials can be delivered to a transfusion bottle one by one by using the same drug mixing and delivery device. This eliminates the need for using a drug mixing and delivery device for each solute vial, thereby simplifying operation and reducing cost.

1-33. (canceled)

34. A drug mixing and delivery device, comprising:
an outer sleeve comprising a first cavity and a second cavity, capable of receiving and engaging a conventional drug vial, separated by a flange;
an inner sleeve, connected to said outer sleeve, comprising a third cavity capable of receiving and engaging a conventional drug vial; and
a needle, connecting said outer sleeve and said inner sleeve, wherein said needle extends through said flange and said inner sleeve.

35. The drug mixing and delivery device of claim 34, wherein said outer sleeve further comprises a collar, formed on an inner wall of said outer sleeve, comprising an annular projection, capable of engaging and securing a conventional drug vial, wherein said collar is adjacent to a circular bulging portion, formed on an inner wall of said outer sleeve, capable of engaging and securing a conventional drug vial.

36. The drug mixing and delivery device of claim 35, wherein said annular projection has a cross-section triangular in shape and wherein an inner diameter of said flange is smaller than a diameter of said circular bulge portion.

37. The drug mixing and delivery device of claim 34, wherein said inner sleeve is positioned between said flange and said circular bulging portion.

38. The drug mixing and delivery device of claim 34, further comprising at least one flexible expansion joint, formed in said outer sleeve, capable of deforming said outer sleeve to engage a drug vial.

39. A drug mixing and delivery device, comprising:
an outer sleeve comprising a first cavity, capable of receiving and engaging a conventional drug vial, and a distance plate;
an inner sleeve, connected to and movable relative to said outer sleeve, comprising a second cavity, capable of receiving and engaging a conventional drug vial, and a distance piece;
a needle, connecting said outer sleeve and said inner sleeve; and
an elastic member positioned between said distance piece and said distance plate.

40. The drug mixing and delivery device of claim 39, wherein said outer sleeve and said inner sleeve comprise retaining members for engaging with each other.

41. The drug mixing and delivery device of claim 39, wherein said elastic member is selected from the group consisting of a spring and an elastic rubber sheath.

42. The drug mixing and delivery device of claim 39, wherein a distal end of said needle, adjacent to said distance piece, is encircled by a protective sheath.

43. The drug mixing and delivery device of claim 39, wherein said inner sleeve further comprises a round bulge formed on an inner wall of said inner sleeve capable of engaging and securing a conventional drug vial.

44. The drug mixing and delivery device of claim 39, wherein the distance piece is positioned at a distal end of said inner sleeve and wherein a diameter of said distance piece is greater than a diameter of said outer sleeve.

45. The drug mixing and delivery device of claim 39, wherein said outer sleeve further comprises expansion joints, formed on an inner wall of said outer sleeve, capable of deforming said outer sleeve to engage a drug vial.

46. The drug mixing and delivery device of claim 39, wherein said outer sleeve further comprises channels and notches formed on an inner wall of said outer sleeve.

47. The drug mixing and delivery device of claim 39, wherein said inner sleeve further comprising clippers capable of engaging with said channels and locking with said notches.

48. A drug mixing and delivery device, comprising:
an outer sleeve comprising:
a first cavity, capable of receiving and engaging a conventional drug vial,
a movable inner support, for positioning said outer sleeve relative to an inner sleeve, and
a bush, comprising a movable plate, for positioning said outer sleeve relative to an inner sleeve;
an inner sleeve, connected to and movable relative to said outer sleeve, comprising a second cavity, capable of receiving and engaging a conventional drug vial;
a needle, connecting said outer sleeve and said inner sleeve; and
at least one elastic member for positioning said inner sleeve relative to said outer sleeve.

49. The drug mixing and delivery device of claim 48, wherein said elastic member comprises a first elastic member positioned between said inner support and said movable plate and a second elastic member positioned between a distal end of said bush and said movable plate.

50. The drug mixing and delivery device of claim 48, wherein said needle is fixed to said movable plate and is positioned between a thorough hole formed in said inner support and a thorough hole formed in said bush.

51. The drug mixing and delivery device of claim 48, wherein said movable plate is confined within said bush by a collar.

52. The drug mixing and delivery device of claim 48, wherein said inner sleeve and said outer sleeve are irremovably connected by a cap connected to said outer sleeve via a removable ripping ring.

53. The drug mixing and delivery device of claim 48, wherein said inner sleeve further comprises an annular step or a bulge, formed on an inner wall of inner sleeve, capable of engaging and securing a conventional drug vial.
